(19) United States
(12) Patent Application Publication
(10) Pub. No.: US 2026/0207128 A1
(43) Pub. Date: Jul. 23, 2026

(54) CONTROL SYSTEM FOR SLEEP MONITORING DEVICE

(71) Applicant: KONINKLIJKE PHILIPS N.V., EINDHOVEN (NL)

(72) Inventors: JUN SHI, SHANGHAI (CN); BIN YIN, SHANGHAI (CN); SHENG JIN, SHANGHAI (CN); GUAN QUN ZHANG, SHANGHAI (CN); JUN ZENG FU, SHANGHAI (CN)

(21) Appl. No.: 19/140,729

(22) PCT Filed: Dec. 13, 2023
(86) PCT No.: PCT/EP2023/085714

§ 371 Date: Jun. 18, 2025

(30) Foreign Application Priority Data
Dec. 21, 2022 (WO) PCT/CN2022/140834

(51) Int. Cl. A61B 5/00; A61B 5/0205; A61B 5/11

(52) U.S. Cl. CPC A61B 5/4818; A61B 5/0205; A61B 5/1102; A61B 5/7225

(57) ABSTRACT

Embodiments of the present disclosure relate to a control system for controlling a sleep monitoring device. The control system determines a sleep position of a user of the sleep monitoring device based on the BCG signal. The control system generates a sequence of physiological parameters from the BCG signal. The control system adjusts configuration for processing the sequence of physiological parameters based on the sleep position. The control system controls the sleep monitoring device to monitor a sleep event of the user based on the sequence of physiological parameters and the adjusted configuration. In accordance with embodiments of the present disclosure the sleep monitoring device can monitor the sleep event with a high quality consistently from BCG signals with improved efficiency.

### FIELD OF THE INVENTION

Embodiments of the present disclosure generally relate to a field of healthcare, and more specially, to a control system for a sleep monitoring device, a sleep monitoring system and a sleep monitoring device.

### BACKGROUND OF THE INVENTION

Sleep monitoring devices like sleep tracking pillows and mattresses can monitor users' physiological parameters (e.g., breath, heartbeat, and etc.) with sensors like Ballistocardiography (BCG) sensor for detecting sleep events such as obstructive sleep apnea (OSA). The BCG sensor is an unobtrusive sensor for mechanical vibrations, and may be embedded in pillows, mattresses, and other wearables to collect heart generated ballistic signal.

Due to the property of the BCG sensor, the signals of BCG sensor are closely related to a sleep position of a user and have different structures. The sleep position also causes different level of noises to the signals. At the same time, the sleep events like OSA are also closely related to the sleep positions, e.g., OSA is more likely to occur at a supine position, but less likely at a prone position. There is a need to improve the sleep monitoring devices in this regard.

### SUMMARY OF THE DISCLOSURE

Sleep positions of a user of a sleep monitoring device induce signal differences to raw BCG signals. The raw BCG signals have different signal patterns at different sleep positions. This may cause the fluctuation of the quality of the physiological output of the sleep monitoring device. At the same time, some sleep related issue like OSA is closely related to the sleep position. It happens more at a supine position than other positions.

In view of the above, embodiments of the present disclosure propose a control system for controlling a sleep monitoring device, a sleep monitoring system and a sleep monitoring device.

In a first aspect, the embodiments of the present disclosure provide a control system for controlling a sleep monitoring device. The control system comprises an input interface configured to receive a Ballistocardiography (BCG) signal; an output interface for communicating with the sleep monitoring device; and a processor coupled to the input interface and configured to determine, from a plurality of predefined sleep positions, a sleep position of a user of the sleep monitoring device based on the BCG signal; generate a sequence of physiological parameters from the BCG signal; adjust configuration for processing the sequence of physiological parameters based on the sleep position; and control, via the output interface, the sleep monitoring device to monitor a sleep event of the user based on the sequence of physiological parameters and the adjusted configuration.

According to embodiments of the present disclosure, the control system adjusts configuration of the sleep monitoring device according to the sleep position of the user, such that the sleep monitoring device monitors the sleep event in a manner specific to sleep position. As an example, the control system may adjust the monitoring interval of the sleep monitoring device, for example, decrease the monitoring interval for the supine position or increase it for the prone position. Then, the sleep monitoring device will monitor the sleep event more frequently at the supine position, and less frequently at the prone position. In this way, the sleep monitoring device can monitor the sleep event with a high quality consistently from BCG signals with improved efficiency.

In some embodiments of the first aspect, the processor may be further configured to determine the sleep position of the user of the sleep monitoring device as follows. The processor is configured to detect a peak in the BCG signal, extract at least one signal pattern from the BCG signal by capturing a portion of the BCG signal with at least one sample window, wherein a center of the at least one sample window is aligned with a time of the peak; determine a set of correlations between the at least one signal pattern and a plurality of patterns for the plurality of predefined sleep positions; and determine the sleep position corresponding to a maximum in the set of correlations. The correlation may indicate the similarities between the signal pattern of the BCG signal and the patterns for different sleep positions. In this way, the sleep position of the user can be determined by comparison between the signal pattern of the BCG signal and the patterns for different sleep positions.

In some embodiments of the first aspect, each of the at least one sample window may have a predefined size corresponding to a range of a physiological parameter. In this way, multiple signal patterns can be obtained for different physiological parameters. For example, the sizes of the sample windows may correspond to different heart rates. When the heart rate higher, the corresponding window size is smaller. The best match between the patterns can be found across all of possible ranges of the heart rates. Thus, the sleep position can be determined precisely in consideration of various ranges of the physiological parameter.

In some embodiments of the first aspect, determining, from the plurality of predefined sleep positions, the sleep position from the plurality of predefined sleep positions comprises: determining a sample window of the at least one sample window corresponding to the maximum in the set of correlations; detect a further peak in the BCG signal; extracting a signal pattern from the BCG signal by capturing a portion of the BCG signal with the sample window, wherein a center of the sample window is aligned with a time of the further peak; determining a further set of correlations between the signal pattern and the plurality of patterns; and determining the sleep position corresponding to a maximum in the further set of correlations. Once the maximum correlation is found, the corresponding sample window can be regarded as an appropriate window matching with the physiological parameters of the user, e.g. heart rate. For the subsequent BCG signal, the control system may use this sample window solely to determine the sleep position promptly and save computing resources.

In some embodiments of the first aspect, the processor is further configured to filter the BCG signal with a predefined filtering approach to enhance features in the BCG signal; and generate the sequence of physiological parameters based on the features in the filtered BCG signal. Before generation of physiological parameters, the predefined filtering approach may be applied as a general approach for all sleep positions. In this way, the quality of the physiological parameters is improved.

In some embodiments of the first aspect, the processor may be further configured to select the predefined filtering approach based on the sleep position; filter the BCG signal with the selected predefined filtering approach to enhance features in the BCG signal; and generate the sequence of physiological parameters from the features in the filtered BCG signal. The BCG signal is closely related to sleep positions and has different patterns at different sleep positions. In this way, the filtering approach is optimized for the specific sleep position. Thus, the features for the sleep position are well recognized and then the physiological parameters are generated with improved quality and less errors.

In some embodiments of the first aspect, wherein the processor may be further configured to generate the sequence of physiological parameters from the features in the filtered BCG signal by, for each of a first set of sliding windows, detecting features of the filtered BCG signal in the sliding window based on a feature detection manner; and generating one or more values of the sequence of physiological parameters based on the detected features, wherein the feature detection manner and a first stride between starting times of two adjacent sliding windows of the first set of sliding windows are adjustable based on the sleep position. The control system may sample the BCG signal with sliding windows and calculate the physiological parameters based on the features in the sampled sliding windows. In addition to the filtering approach, it selects the corresponding feature detection manner and the stride that are optimized for the specific sleep position. In this way, the quality of physiological parameters is improved.

In some embodiments of the first aspect, the processor may be further configured to control the sleep monitoring device to monitor the sleep event of the user by determining a variability of the sequence of physiological parameters; and responsive to the variability being lower than a predetermined threshold, determining an occurrence of the sleep event. When the sleep event occurs, the amplitude of physiological parameters will significantly decrease. In this way, the sleep event can be detected by monitoring the variability of the physiological parameters.

In some embodiments of the first aspect, the processor may be further configured to adjust the configuration for processing the sequence of physiological parameters by adjusting a second stride of a second set of sliding windows based on the sleep position, wherein the second stride indicates a difference between starting times of two adjacent sliding windows of the second set of sliding windows, wherein the processor may be further configured to control the sleep monitoring device to monitor the sleep event based on at least a portion of the sequence of physiological parameters within at least a part of the second set of sliding windows. The sleep monitoring device is controlled to monitor the sleep event within each of the sliding windows. The stride of the sliding windows is optimized for the specific sleep position. In this way, the sleep monitoring device can monitor the sleep event promptly and precisely and in the meantime save energy and storage.

In some embodiments of the first aspect, the processor may be further configured to: responsive to a detection of the sleep event by the sleep monitoring device, decrease the second stride between two adjacent sliding windows; and/or responsive to no detection of the sleep event by the sleep monitoring device, increase the second stride between two adjacent sliding windows. In this way, the stride, and thus the monitoring interval for the sleep events may be adjusted dynamically for one sleep position.

In some embodiments of the first aspect, the processor is further configured to adjust a size of each of the second set of sliding windows based on the sequence of physiological parameters of the user. In this way, the size of the windows for monitoring the sleep event may be adjusted to match the current physiological parameters of the user. For example, the size of the windows may be a predefined multiple of the heartbeat cycle of the user.

In some embodiments of the first aspect, the processor may be further configured to determine occurrence number of the sleep event for one or more of the detected sleep positions during a period of time; and adjust the second stride of the second set of sliding windows based on the occurrence number. In this way, the control system may dynamically adjust the monitoring interval of the sliding windows for different sleep positions. For example, if a sleep position counts for the highest proportion of all sleep events, the corresponding stride for that sleep position can be reduced.

In some embodiments of the first aspect, the processor is further configured to adjust a sampling frequency of a BCG sensor for sensing the BCG signal and/or a data storage manner of the BCG signal based on the sleep position. In this way, the sampling frequency and the data storage manner of the BCG sensor may be optimized for the specific sleep position for energy and storage saving.

In a second aspect, the embodiments of the present disclosure provide a sleep monitoring system, comprising: a Ballistocardiography (BCG) sensor configured to generate a BCG signal; a sleep monitoring device configured to monitor a sleep event of a user; and the control system in the first aspect of the present disclosure, wherein the BCG sensor is communicatively coupled to an input interface of the control system, and the sleep monitoring device is communicatively coupled to an output interface of the control system.

In a third aspect, the embodiments of the present disclosure provide a sleep monitoring device, comprising: a processing unit; and a memory having a plurality of instructions stored thereon, the plurality of instructions, when executed by the processing unit, cause the processing unit to determine, from a plurality of predefined sleep positions, a sleep position of a user of the sleep monitoring device based on a Ballistocardiography (BCG) signal; generate a sequence of physiological parameters from the BCG signal; adjust configuration for processing the sequence of the physiological parameters based on the sleep position; and monitor a sleep event of the user based on the sequence of physiological parameters and the adjusted configuration.

In a forth aspect, the embodiments of the present disclosure provide a method for controlling a sleep monitoring device, comprising: determining, from a plurality of predefined sleep positions, a sleep position of a user of a sleep monitoring device based on a Ballistocardiography (BCG) signal; generating a sequence of physiological parameters from the BCG signal; adjusting configuration for processing the sequence of physiological parameters based on the sleep position; and controlling the sleep monitoring device to monitor a sleep event of the user based on the sequence of physiological parameters and the adjusted configuration.

In a fifth aspect, the embodiments of the present disclosure provide a computer program product comprising a computer readable medium. The computer readable medium has computer readable code embodied therein. The computer readable code is configured such that, on execution by a processor, the processor is caused to perform the method of the fourth aspect.

It is to be understood that the Summary is not intended to identify key or essential features of embodiments of the present disclosure, nor is it intended to be used to limit the scope of the present disclosure. Other features of the present disclosure will become easily comprehensible through the description below.

### BRIEF DESCRIPTION OF THE DRAWINGS

The details of one or more embodiments of the present disclosure are set forth in the accompanying drawings and the description below. Other features, aspects, and advantages of the disclosure will become apparent from the description, the drawings, and the claims, wherein:

FIG. 1 is a block diagram illustrating an example sleep monitoring system in accordance with an example embodiment of the present disclosure;

FIG. 2A is a graph illustrating example BCG signals at different sleep positions;

FIG. 2B is a graph illustrating example BCG signal segment with typical peaks and troughs;

FIG. 3 is a block diagram illustrating an overview of modules in the sleep monitoring device in accordance with an example embodiment of the present disclosure;

FIG. 4 is a flowchart illustrating an example process for controlling a sleep monitoring device in accordance with an example embodiment of the present disclosure;

FIG. 5 is a flowchart illustrating an example process for determining a sleep position of a user in accordance with an example embodiment of the present disclosure;

FIG. 6 is a diagram illustrating example patterns of different sleep positions;

FIG. 7 is a flowchart illustrating an example process for generating a sequence of physiological parameters in accordance with an example embodiment of the present disclosure;

FIG. 8 is a diagram illustrating sliding windows for generating physiological parameters;

FIG. 9 is a graph illustrating an example sequence of physiological parameters when a sleep event occurs; and

FIG. 10 is a diagram illustrating sliding windows for monitoring a sleep event.

Throughout the figures, same or similar reference numbers will always indicate same or similar elements.

### DETAILED DESCRIPTION OF EMBODIMENTS

Principle of the present disclosure will now be described with reference to some example embodiments. It is to be understood that these embodiments are described only for the purpose of illustration and help those skilled in the art to understand and implement the present disclosure, without suggesting any limitations as to the scope of the disclosure. The disclosure described herein can be implemented in various manners other than the ones describe below.

As used herein, the term “comprise/include” and its variants are to be read as open terms that mean “comprise/include, but not limited to.” The term “based on” is to be read as “based at least in part on.” The term “one embodiment” and “an embodiment” are to be read as “at least one embodiment.” The term “another embodiment” is to be read as “at least one other embodiment.” Moreover, it is to be understood that in the context of the present disclosure, the terms “first,” “second” and the like are used to indicate individual elements or components, without suggesting any limitation as to the order of these elements. Further, a first element may or may not be the same as a second element.

As mentioned above, sleep monitoring devices can monitor sleep events unobtrusively with BCG sensors. Due to the property of the BCG sensors, the signals of BCG sensors are closely related to a sleep position of a user, and the sleep event like OSA is also closely related to the sleep position. Among four sleep positions, right lateral, left lateral, supine, and prone, most of OSA events occur at a supine position, while least occur at a prone position. However, the current sleep monitoring devices analyses the BCG signals in a uniform manner, regard less of the user's sleep position. Therefore, the sleep monitoring devices wastes processing or storage resources when the sleep event is less likely to happen, e.g., at a prone position, but cannot detect the sleep event promptly and precisely when it is more likely to happen, e.g., at a supine position.

To address these and other potential problems, embodiments of the present disclosure provide a solution for controlling the sleep monitoring device. According to the embodiments of the present disclosure, the sleep monitoring device can monitor the sleep event with a high quality consistently and with improved efficiency

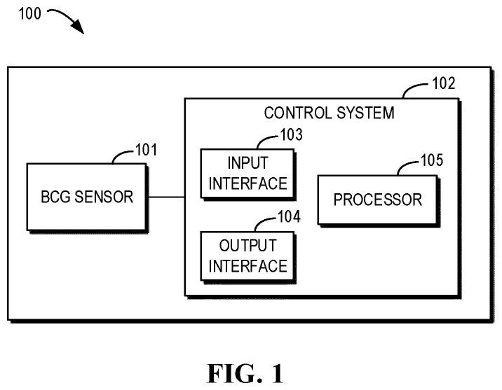

Reference is now made to FIG. 1 which illustrates an example sleep monitoring device 100 in accordance with an example embodiment of the present disclosure. The sleep monitoring device 100 may be implemented in, for example, a sleep monitoring pillow, a mattress or other wearable devices or portable devices.

As shown, the sleep monitoring device 100 includes a BCG sensor 101 and a control system 102. The BCG sensor 101 is responsible for collecting the heart generated ballistic signals of a user of the sleep monitoring device 100. The BCG sensor 101 may be an unobtrusive sensor, for example, a BCG foil for sensing mechanical vibrations caused by the user's heart activities. The signal is in the 1-20 Hz frequency range which is caused by the mechanical movement of the heart and can be recorded by noninvasive methods. In some embodiments, the BCG sensor 101 may generate raw BCG signals by sampling the sensed mechanical vibrations at a specified frequency and quantizing the sampled data with a specified number of bits. Therefore, the BCG sensor 101 can generate digital BCG signals for further processing. In FIG. 1, the BCG sensor 101 is illustrated as being included in the sleep monitoring device 100. In some embodiments, the BCG sensor 101 may be an external device to the sleep monitoring device 100.

The BCG sensor 101 is communicatively coupled to the control system 102. The control system 101 includes an input interface 103, an output interface 104, and a processor 105. The input interface 103 may be configured to receive the BCG signal from the BCG sensor 101. The output interface 104 may be configured to output signals for controlling the BCG sensor 101 and the sleep monitoring device 100. The processor 105 may execute computer programs or instructions stored in a memory. The memory may be included in or external to the control system 102. When the processor 105 executes the computer programs or the instructions, the control system 102 can perform methods according to the embodiments of the disclosure to control, via the output interface 104, operations of the sleep monitoring device 100. In some embodiments, the processor 105 may adjust one or more configurations of the sleep monitoring device 100 based on the determined sleep position.

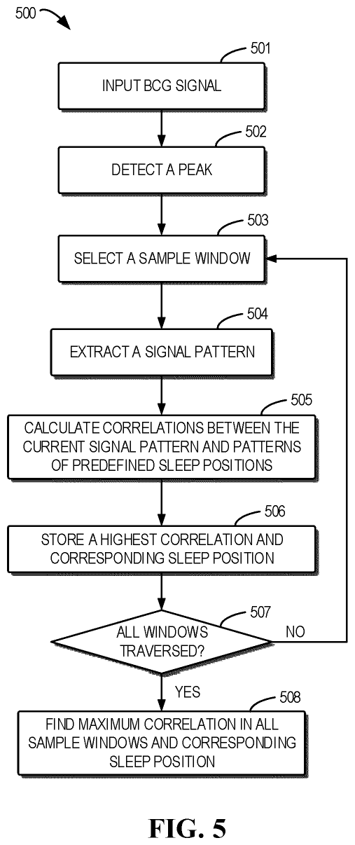

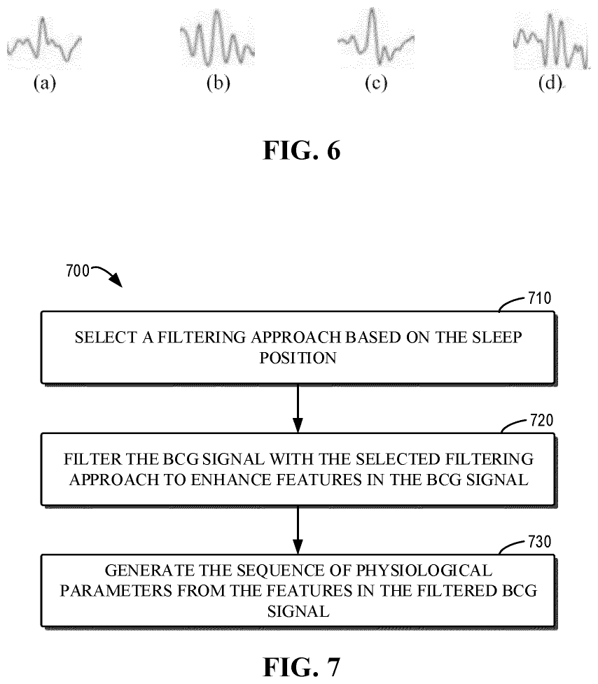

Specially, the control system 102 may process the received BCG signal to determine the current sleep position of the user. The control system 102 may compare the signal pattern of the BCG signal with a set of pattern templates for different positions. The determination of the sleep position will be described in more detail with reference to FIGS. 5 and 6. In some embodiments, the control system 102 may generate physiological parameters of the user based on the BCG signal. The physiological parameters may include, for example, heart or respiration related features, such as heart rate, respiration rate, respiration amplitude, or other physiological parameters derived from the above parameters, e.g. heart rate variability (HRV). In some embodiments, the control system 102 may detect the features in the BCG signals, e.g., peaks, to calculate the physiological parameters based on the features in the BCG signal.

By use of the physiological parameters, the sleep monitoring device 100 detects a possible sleep event underlying the raw BCG signal. In some embodiments, the sleep monitoring device 100 may continuously processes the the physiological parameters derived to detect whether a sleep event occurs. The sleep event may be a sleep disorder, for example, an apnea, OSA, and the like. For example, the sleep monitoring device 100 may identify the sleep event when it detects a significant decrease of the physiological parameters for a period of time. In some embodiments, the physiological parameters may be represented as a time sequence. The sleep monitoring device 100 may apply a set of sliding windows to the time sequence, monitor the physiological parameters in the time sequence and determine whether the sleep event exists in each of the sliding windows. In a sliding window, when the amplitude variance is less than a certain threshold, it is regarded as there is a sleep event from within the sliding window. As mentioned above, the probability of the occurrence of sleep event is closely related to the sleep position. In some embodiments, the stride of the sliding windows may be dynamically adjusted according to the sleep position of the user.

Although the example sleep monitoring device is shown and described with reference to FIG. 1, it is to be understood and appreciated that the sleep monitoring device is not limited by the above description with reference to FIG. 1. It is to be understood and appreciated that the sleep monitoring device according to the disclosure may comprise additional components that are not shown, and some of the components in FIG. 1 may be omitted.

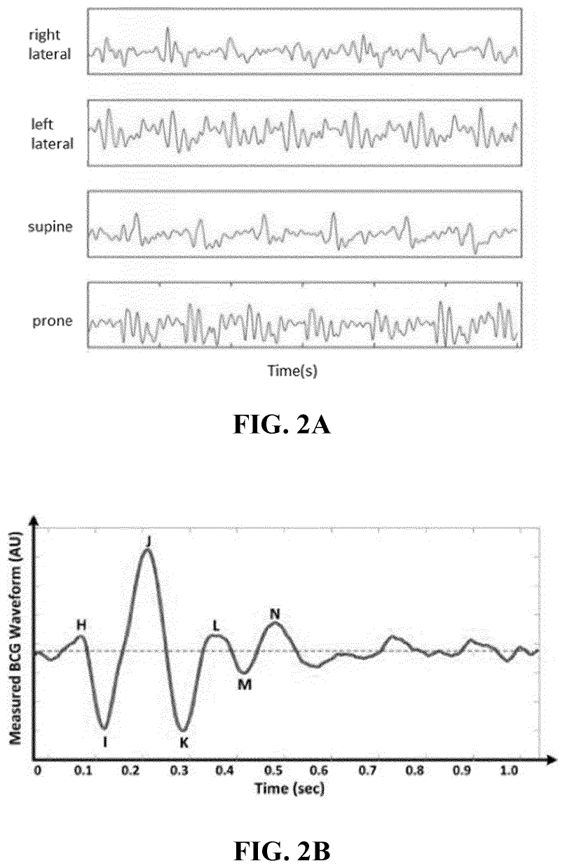

FIG. 2A is a graph illustrating example BCG signals at different sleep positions. As shown, sleep positions induce structural signal differences to the raw BCG signals. The BCG signals at different sleep positions have different signal patterns. For example, the BCG signal at the supine position has a peak (e.g., the J peak) in a single heartbeat cycle which is significantly high than other peaks, while the BCG signal at the prone position has neighboring high peaks (e.g., the H and J peaks) in a signal heartbeat cycle.

FIG. 2B is a graph illustrating example BCG signal segment with typical peaks and troughs. The peaks and troughs include HJKLMN having corresponding amplitudes. Normally, the J peak is the highest one. However, at different sleep positions the relative amplitudes of HIJKLMN are different, as shown in FIG. 2A. Even for some cases the J peak is not the highest one, i.e. amplitude of H may be higher than amplitude of J, and a real J peak should be identified. At the same time, due to the difference of heart rate, JJ interval varies for different person and at different situation. It may introduce lots of errors for peak detection and IJK complex identification, which may degrade the output quality of the sleep monitoring device.

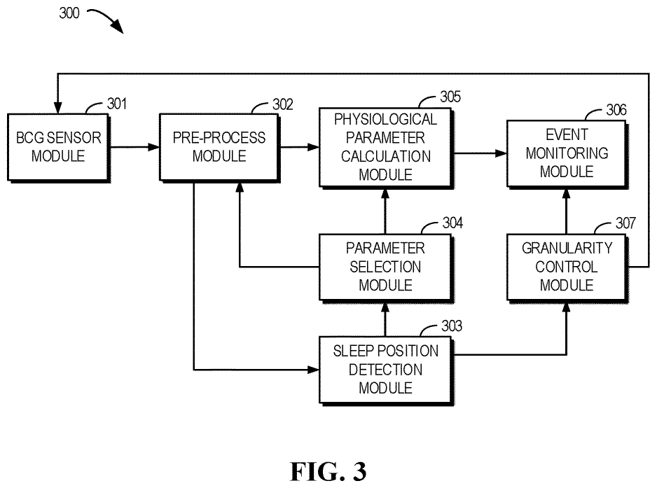

FIG. 3 is a block diagram illustrating an overview of modules in the sleep monitoring device in accordance with an example embodiment of the present disclosure.

The BCG sensor module 301 is responsible for collecting the raw BCG signals of the user. In some embodiments, the BCG sensor module 301 may generate raw BCG signals by sampling the sensed mechanical vibrations at a specified frequency and quantizing the sampled data with a specified number of bits. The BCG sensor module 301 may continuously output the raw BCG signals to the pre-process module 302.

Due to the property of the BCG sensor, the raw BCG signal normally contains high frequency noise. At the same time baseline wander and respiration modulation also are a source of noise coupled with raw BCG signals. The pre-process module 302 is responsible for removing the high frequency noises, the baseline wander and inhalation and exhalation modulation. High frequency noises may be reduced or removed through band pass filters. Baseline wander and respiration modulation may be reduced or removed through differencing. In one embodiment, the pre-process module 302 can be implemented as a filter.

With reference to FIGS. 2A and 2B, in the BCG signal at a prone position, amplitudes of the J peak and H peak are close. If the sleep monitoring device calculates the physiological parameters (e.g., heart rate) based on identification of the J peaks in the BCG signal, it would output the physiological parameters with more errors.

In some embodiments, after high frequency noises, the baseline wander and the respiration modulation are removed or reduced, the pre-processing module 302 may further be configured to filter the BCG signal to enhance the features, e.g., the peaks (IJK peaks), such that the calculation of the physiological parameters would be more precise. For example, the pre-process module 302 may further include another filter (i.e., sleep position related filter) with a predefined filtering approach that is applicable to all sleep positions. After the sleep position is determined, the predefined filtering approach will be adjusted according to the determined sleep position. This another filter can be alternatively placed at a different module, e.g., physiological parameter calculation module 305.

At the beginning, the sleep position has not been determined yet, in this case, the pre-process module 302 may filter the BCG signal with the predefined filtering approach as default, which is applicable to all kinds of the sleep positions. After the sleep position has been determined, the pre-process module 302 may replace the default filtering approaching with a new position-specific filtering approach. Afterward, the pre-process module 302 outputs the processed BCG signal to the physiological calculation module 305 for generation of physiological parameters of the user. In the meantime, the pre-process module 302 outputs the processed BCG signal to the sleep position detection module 303.

The sleep position detection module 303 is responsible for detecting the current sleep position of the user of the sleep monitoring device. The sleep position may be one of a plurality of predefined sleep positions including right lateral, left lateral, supine and prone. The sleep position detection module 303 may detect the sleep position through pattern recognition where the signal pattern of the BCG signal is compared with pattern templates for the predefined sleep positions. The sleep position detection module 303 may determine one of the predefined sleep positions to be the user's sleep position. The determined sleep position has the most matched pattern to that of the BCG signal. The position detection process will be described in more detail with reference to FIG. 5 and FIG. 6.

The parameter selection module 304 is responsible for selecting optimized parameters for different sleep positions. Based on the feedback of the sleep position detection module 303, the parameter selection module 304 may select necessary parameters needed for physiological parameter calculation. These parameters may be trained through population data or may be personalized data that trained for current user.

The parameters may include filtering approaches specific to different positions. The parameter selection module 304 may communicate with the pre-process module 302 to inform the selected filtering approach so as to enhance quality of the BCG signal for physiological calculation. In some embodiments, the sleep position related filter for filtering the BCG signal can be performed at the physiological parameter calculation module 305.

The filtering approach has a filtering shape to allow or suppress certain portions of the signal in frequency. An ideal filter would have unit gain (0 dB) in its pass band and a gain of zero (−infinity dB) in its stop band. It would pass only the required frequencies without adding or subtracting anything from the signal. In real world, there is no perfect filters in the real word, and some amount of non-unity gain in the pass band (e.g. insertion loss and pass band ripple) and non-zero gain in the stop band (e.g. finite stop band attenuation and stop band ripple) are acceptable. With different designs, there are different types or shapes of a filter, i.e. different filter models. The filtering models may include Gaussian filter, Kalman filter, and etc. In some embodiments, the filtering approach defines a type of a filter and corresponding parameters.

The parameter selection module 304 also selects parameters for generation of physiological parameters of the user and communicates the selected parameters to the physiological calculation module 305 which may use a set of sliding windows to calculate values of the physiological parameters in each of the sliding windows.

The parameters for generating physiological parameters may indicate, for example, features detection manners, sliding window sizes and stride sizes for different sleep positions. In some embodiments, the parameters may include position-specific peak thresholds for detecting peaks in the BCG signal. As an example without limitation, for the left lateral and supine positions, the peak threshold may be set as 60% of the maximum amplitude of the signal in the window. For the right lateral position, the peak threshold may be 80% of the maximum amplitude in the window. For the prone position, the threshold is set to 85% of the maximum amplitude.

The physiological parameter calculation module 305 is responsible for calculating the physiological parameters like heart rate and respiration rate, etc. through input signals. The physiological parameter calculation module 305 uses a set of sliding windows to calculate the values of the physiological parameters in each sliding windows. In some embodiments, the size and the stride of the sliding windows also depends on the current sleep position of user. The physiological parameter calculation module 305 outputs a sequence of the calculated physiological parameters to the event monitoring module 306 for sleep event detection.

The event monitoring module 306 is responsible for monitoring the physiological issues of the user, e.g., OSA event monitoring. There are several different ways to implement OSA event monitoring, e.g., through breathing events or through BCG signals. An apnea can be identified as a decrease in airflow by e.g., 80% of baseline for at least 10 seconds. When sleep-related breathing events occur, the user's breathing amplitude will significantly decrease, which will lead to the drop of amplitude of the physiological parameters derived from the BCG signal as well. In some embodiments, amplitude variance may be used to detect the sleep event.

The event monitoring module 306 may apply another set of sliding windows to the input sequence of physiological parameters to continuously monitor the sleep event. The size of the sliding windows may depend on physiological parameters, e.g. heart rate or respiration rate. The stride of the sliding windows is indicative of a difference between starting times of two adjacent windows. In some embodiments, the stride depends on the current sleep position.

For each of the sliding windows, the event monitoring module 306 may examine the variability of the sequence of physiological parameters to determine whether the sleep event occurs. For example, when the amplitude variance in a sliding window (more than 10 s) is less than certain threshold, it is regarded as there is a sleep event within the sliding window. The event monitoring module 306 may combine continuous events to eliminate redundant counting.

On average, a sleep event occurs most in the supine position among the four positions and occurs least in the prone position. Based on this fact, configuration of the sleep monitoring device may be adjusted accordingly. The granularity control module 307 is responsible for adjusting the configuration of the sleep monitoring device. In some embodiments, the granularity control module 307 may adjust the stride of sliding windows of the event monitoring module 306 based on the sleep position. The granularity control module 307 may store the strides for different sleep positions. The strides may be based on population data or customized for a particular user. For example, the stride may be set as 5 seconds for the supine position, 10 seconds for left lateral position, 20 seconds for right lateral position, and 60 for the prone position.

In some embodiments, the stride may be adjusted dynamically for one sleep position. For example, for left lateral position a stride of 10 seconds is initially selected. When there is detected sleep event, the next stride may be changed to 5 seconds. If no event is detected, the stride may be increased to 7 seconds and then 10 seconds again for the following monitoring window until a new event is detected.

In some embodiments, the stride may be dynamically adjusted based on the user's historical sleep events during a period of time. For example, the number of sleep events for the current user at different sleep positions may be recorded daily and the stride is adjusted according to the percentage of the one sleep position in all four positions. For example, for a particular user the supine position may count for 100% of the sleep event. Then the stride for the supine position may be changed to a smaller value and the strides for all other positions may be changed to larger values.

Besides adjust the strides for the event monitoring module 306, the granularity control module 207 may feed the information to the BCG sensor module 301 to adjust the raw data collection settings based on the sleep position. The raw data collection settings may include the sampling frequency and the precision of sampled data (e.g., the bit depth). For example, for the supine position a highest sample frequency 200 Hz may be used for data collection. For the left lateral and right lateral positions, the sample frequency may be 100 Hz and 50 Hz. For the prone position, the sample frequency may be 30 Hz. Similarly, more bits may be used to represent the sampled data at the supine position, less bits for the left lateral and the right lateral, and least bits for the prone position.

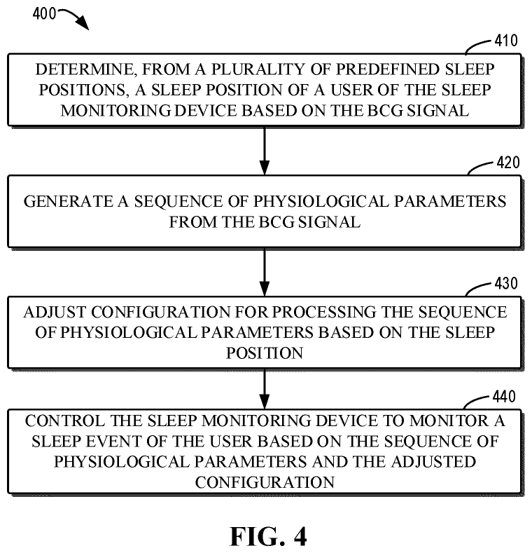

FIG. 4 is a flowchart illustrating an example process for controlling a sleep monitoring device in accordance with an example embodiment of the present disclosure. While the methodologies are shown and described as being a series of acts that are performed in a sequence, it is to be understood and appreciated that the methodologies are not limited by the order of the sequence. For example, some acts can occur in a different order than what is described herein. In addition, an act can occur concurrently with another act. Further, in some instances, not all acts may be required to implement a methodology described herein.

Moreover, the acts described herein may be computer-executable instructions that can be implemented by one or more processors and/or stored on a computer-readable medium or media. The computer-executable instructions may include a routine, a sub-routine, programs, a thread of execution, and/or the like. Still further, results of acts of the methodologies may be stored in a computer-readable medium, displayed on a display device, and/or the like. In one embodiment, the process 400 may be implemented by the sleep monitoring system 100, in particular, by the control system 102 shown in FIG. 1. For understanding of embodiments of the disclosure, FIG. 4 is described with reference to FIGS. 1-3.

At block 410, the control system 102 determines, from a plurality of predefined sleep positions, a sleep position of a user of the sleep monitoring device based on a BCG signal. The predefined sleep positions may include right lateral, left lateral, supine, and prone positions. One of the predefined sleep positions is determined to be the current sleep position of the user of the sleep monitoring device 100.

The BCG sensor 101 of the sleep monitoring device 100 gathers mechanical vibrations caused by the user of the device and transmits via the input interface 103 a raw BCG signal to the control system 102.

As pre-processing, the control system 102 may first remove noises from the raw BCG signal, including high frequency noises and noises caused by baseline wander and respiration modulation. For example, the control system 102 may filter the raw BCG signal with a band pass or low pass filter to remove the high frequency noises, and applying differencing methods to remove the baseline wander and respiration modulation. Before determining the sleep position, the control system 102 may further apply a general filter to enhance features of the pre-processed signal, e.g., various peaks and troughs, such as the IJK complex. By doing this, the features would be easily detected and errors in detecting the features would be reduced. The general filter is suitable for all of the predefined sleep positions, and may be replaced with position-specific filters afterwards.

In some embodiments, the control system compares the signal pattern of the BCG signal with pattern templates of the predefined position to determine the current sleep position of the user. A sleep position having the most similar template with the signal pattern of BCG signal is determined to be the current sleep position.

At block 420, the control system 102 generates a sequence of physiological parameters from the BCG signal. The physiological parameters may be one or more of heart rate, respiration rate, and amplitude of respiration that vary over time. The control system 102 may use sliding window methods to calculate the physiological parameters.

In particular, the control system may apply a set of sliding windows to the BCG signal and calculates one or more values of the physiological parameters with respect to each of the sliding window. For example, for each window spanning multiple heart cycles, the control system 102 detects features (e.g., J peaks) in the BCG signal to calculate an average of the heart rate. Alternatively, the control system may demodulate the BCG signal by applying a low-pass filter to obtain a respiration signal, and detects peaks in the respiration signal to calculate respiration rate and amplitude.

At block 430, the control system 102 adjusts configuration for processing the sequence of physiological parameters based on the sleep position. In some embodiments, the sleep monitoring device may be configured to continuously monitor physiological parameters to detect sleep events like apnea or OSA. For example, the control system 102 may apply another set of sliding windows to the sequence of physiological parameters, and determines whether the sleep event occurs by analyzing physiological data in each sliding window. The configuration may include a stride of the sliding windows which indicates a difference between the starting times of two adjacent sliding windows. Thus, the stride is indicative of how frequently the sleep event is monitored. In some embodiment, for a supine position where the sleep event (e.g., OSA) is more likely to happen, the stride may be adjusted to a smaller value, and for a prone position where the sleep event is less likely to happen, it is adjusted to a larger value.

At block 440, the control system 102 controls, via the output interface, the sleep monitoring device to monitor the sleep event of the user based on the sequence of physiological parameters and the adjusted configuration. Since configuration has been adjusted according to the current sleep position, the sleep monitoring device can monitor the sleep event more efficiently and save the energy and data storage.

FIG. 5 is a flowchart illustrating an example process for determining a sleep position of a user in accordance with an example embodiment of the present disclosure. The example process 500 is example implementation of block 410 of process 400, for example, it is may be implemented at the sleep position detection module 303.

At block 501, the BCG signal is input to the sleep detection module 303. The BCG input signal may be a pre-processed signal with reduced noises.

At block 502, the sleep position detection module 303 detects a peak in the BCG signal. For example, the sleep detection position module 303 may detect a maximum of the BCG signal in a time interval. The time interval is large enough to include one or more heartbeat cycles.

At block 503, the sleep position detection module 303 selects a sample window from a set of sample windows. The sample windows may have different sizes corresponding to respective ranges of a physiological parameter, e.g. the heartbeat cycle. For example, the sample windows may have sizes of 101, 151, 201, and 251 (number of samples over time), where a sample window with a size of 101 corresponds to a highest heart rate, and a sample window with a size of 251 corresponds to a lowest heart rate. As such, the set of sample windows may cover the whole range of human's heart rate.

At block 504, the sleep position detection module 303 extracts a signal pattern from the BCG signal. In some embodiments, it aligns a center of the selected sample window with a time of the detected peak at block 502, and captures a segment of the BCG signal in the sample window. The segment of BCG is regarded as the signal pattern of the BCG signal.

At block 505, the sleep position detection module 303 calculates correlations between the current signal pattern and patterns of predefined sleep positions. The sleep position detection module 303 may locally store the patterns of predefined sleep positions as pattern templates. These stored pattern templates may be obtained through population data or may be personalized for a particular user.

FIG. 6 is a diagram illustrating example patterns of different sleep positions. From left to right the example patterns correspond to left lateral, right lateral, supine and prone correspondingly. In some embodiments, the correlations may be calculated based on the similarities between the current pattern and the pattern templates. For example, the sleep position detection module 303 may calculate the correlations by use of dynamic time warping (DTW) methods.

At block 506, the sleep position detection module 303 stores a highest correlation and the corresponding sleep position. For the current sample window (i.e., the corresponding range of heart rate), a best matched sleep position is determined.

At block 507, it is checked whether all sample windows have been traversed. If a sample window has not been selected yet, the process 500 goes back and repeats actions of blocks 502 to 506; otherwise, the process 500 proceeds to block 508.

At block 508, the sleep position detection module 303 finds the maximum correlation in all sample windows and corresponding sleep position. Once the maximum correlation is found, the corresponding sample window is also determined. The sleep position detection module 303 may use this sample window for determining the sleep position instead of traversing all possible windows.

Specially, the sleep position detection module 303 detects a further peak (e.g., a highest peak in another time interval), and extracts a further signal pattern from the BCG signal by capturing a segment of the BCG signal with the sample window that corresponds to the maximum correlation. Similarly, the center of the sample window may be aligned with the further peak. Then, the sleep position detection module 303 again calculates correlations between the further signal pattern and the patterns for different sleep positions, and determines the sleep position corresponding to a maximum of the correlations.

FIG. 7 is a flowchart illustrating an example process for generating a sequence of physiological parameters in accordance with an example embodiment of the present disclosure. The example process 700 is example implementation of block 420 of process 400.

At block 710, the control system 102 selects a filtering approach based on the sleep position. Based on the feedback of sleep position detection module 304, the parameter selection module 304 of the control system 102 may select a suitable filtering approach for the sleep position. The filtering approach may specify a type of a filter and corresponding filtering parameters.

At block 720, the control system 102 filters the BCG signal with the selected filtering approach to enhance features in the BCG signal. In some embodiments, the pre-process module 302 of the control system 102 may use the selected filtering approach to replace a predefined filtering approach which is applicable to all sleep positions. After the sleep position specific enhancement, the features of the BCG signal would be detected more easily and errors in the detection would be reduced.

At block 730, the control system 102 generates the sequence of physiological parameters from the features in the filtered BCG signal. In some embodiments, the physiological parameter calculation module 305 of the control system 102 may generate the sequence of physiological parameters with a set of sliding windows.

The physiological parameters may be calculated from features in the BCG signal. For example, heart rate may be calculated through counting J peaks in certain time window, e.g. 1 minute or 10 seconds etc. A set of sliding windows could be applied to generate a sequence of physiological parameters continuously. For example, when a one-minute window is used and 66 J peaks are detected in this window, the heart rate in the window is calculated as 66 bpm (beats per minute). The next window may be e.g. one second ahead and 65 J peaks are detected, and the corresponding heart rate is calculated as 65 bpm. In this way, physiological parameter calculation module 305 may obtain a continuous heart rate values in every second interval. A larger window size has a better precision but may cause a delay for physiological parameter calculation. The window size and stride of sliding windows may be selected according to the specific application scenarios.

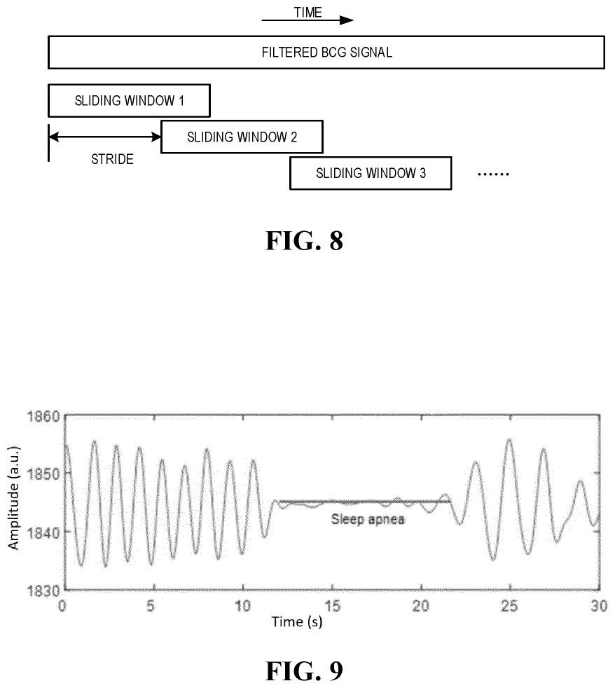

FIG. 8 is a diagram illustrating sliding windows for generating physiological parameters. The sliding windows are arranged along the time axis of the filtered BCG signal. The stride between starting times of two adjacent sliding windows is indicative of how frequently the physiological parameters are calculated. The sliding windows may overlap in time. With a smaller stride size, a dense sequence is obtained, and with a longer stride size, a sparse sequence is obtained. For each sliding window the physiological parameter calculation module 305 calculates one or more values of the sequence of physiological parameters. As such, it may generate the sequence of physiological parameters. The stride may be adjustable based on the sleep position. For example, the stride for the supine position may be set to be the smallest one among all positions, and the stride for the prone position may be set to be the largest one, or otherwise. The present disclosure has no limitations in this regard.

Specially, for each sliding window, the physiological parameter calculation module 305 may detect features, e.g., a particular peak like J peak, of the filtered BCG signal in the sliding window based on a feature detection manner. The feature detection manner may specify a peak detection algorithm and corresponding parameters. In some embodiments, the feature detection manner may be adjustable based on the sleep position. As an example, for the left lateral and supine positions, the peak threshold may be set as 60% of the maximum amplitude of the signal in the window. For the right lateral position, the peak threshold may be 80% of the maximum amplitude in the window. For the prone position, a threshold is set to 85% of the maximum amplitude.

At the same time a neighbor comparison method may be used to find and keep the highest peak in certain range (e.g., in 50 samples) as the real peak. For example, in signals at the prone position, the double peak could be found, and the left peak is kept as the real peak.

FIG. 9 is a graph illustrating an example sequence of physiological parameters when a sleep event occurs. The horizontal axis represents time in seconds, and the vertical axis represents amplitude in arbitrary units. As shown, when a sleep-related breathing events like a sleep apnea occurs, the user's breathing amplitude significantly decreases, which will lead to the drop of the amplitude of physiological parameters as well. The small variations in amplitude caused by the sleep event can be regarded as structural changes of the BCG signal.

In some embodiments, the control system 102 may determine a variability of the sequence of physiological parameters. Specially, the event monitoring module 306 may determine a variability of the sequence of physiological parameters, and responsive to the variability being lower than a predetermined threshold, it determines an occurrence of the sleep event. The event monitoring module 306 detects the occurrence of the sleep event by use of a set of sliding windows as monitoring windows.

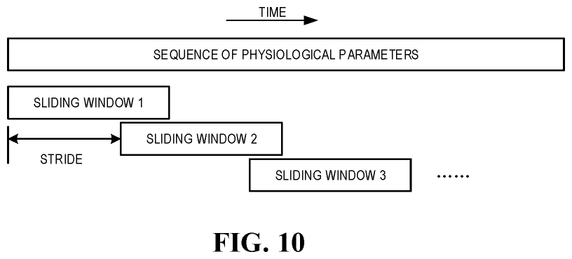

FIG. 10 is a diagram illustrating sliding windows for monitoring a sleep event. The sliding windows are arranged along the time axis of the sequence of physiological parameters. The stride between starting times of two adjacent sliding windows is indicative of how frequently the sleep event is monitored. The sliding windows may overlap in time. In a single window (e.g., at least 10 seconds), when the amplitude variance is less than certain threshold, the event monitoring module 306 determines that there is a sleep event within the window.

In some embodiments, the control system 102 may adjust a size of each of the sliding windows based on the sequence of physiological parameters of the user. The event monitoring module 306 may set the size of each sliding window to be a certain multiple of a heartbeat cycle of the user.

In some embodiments, the control system 102 may also adjust the stride of the windows for monitoring the sleep event based on the sleep position. The granularity control module 307 of the control system 102 may receive information about the sleep position from the sleep position detection module 303, and select a corresponding stride for that sleep position. The strides for different sleep positions may be obtained based on population data or customized for a particular user.

In some embodiments, the control system 102 may adjust the stride dynamically. The event monitoring module 306 may record occurrence numbers of the sleep event for each of the sleep positions during a period of time, and adjust the stride for each sleep position based on the occurrence numbers. For example, when a sleep position, for example supine, counts for the most percentage of all detected events, the stride for that position will be decreased.

In some embodiments, the control system 102 may adjust the stride based on detection results. Responsive to a detection of the sleep event, the controls system 102 may decrease the stride, and responsive to no detection of the sleep event, it may increase the stride. For example, for left lateral position a stride of 10 seconds is initially selected. If a sleep event is detected, the stride for subsequent windows is changed to 5 second. Afterward, if no events detected for a period, the stride may be increased to 7 seconds, and then 10 seconds again for the following monitoring until a new sleep event is detected.

The control system 102 may further control the BCG sensor 101 based on the sleep position of the user. In some embodiments, the granularity control module 307 may adjust a sampling frequency of a BCG sensor for sensing the BCG signal. For example, it may increase the sampling frequency to obtain more data for the supine position where the sleep event is more likely to occur, and decrease the sampling frequency to obtain fewer data for the supine position where the sleep event is less likely to occur. For example, the granularity control module 307 may also increase the bit depth for the supine position to improve data precision, and decrease the bit depth for the prone position to save the data storage of the sleep monitoring device

For the purpose of illustrating spirit and principle of the present disclosure, some specific embodiments thereof have been described above. In general, the various example embodiments may be implemented in hardware or special purpose circuits, software, logic or any combination thereof. Some aspects may be implemented in hardware, while other aspects may be implemented in firmware or software which may be executed by a controller, microprocessor or other computing device. While various aspects of the example embodiments of the present disclosure are illustrated and described as block diagrams, flowcharts, or using some other pictorial representation, it will be appreciated that the blocks, apparatus, systems, techniques or methods described herein may be implemented in, as non-limiting examples, hardware, software, firmware, special purpose circuits or logic, general purpose hardware or controller or other computing devices, or some combination thereof. It is to be understood that the numbers used in examples described herein are just for instance without limiting the disclosure.

In the context of the present disclosure, a machine readable medium may be any tangible medium that can contain, or store a program for use by or in connection with an instruction execution system, apparatus, or device. The machine readable medium may be a machine readable signal medium or a machine readable storage medium. A machine readable medium may include but not limited to an electronic, magnetic, optical, electromagnetic, infrared, or semiconductor system, apparatus, or device, or any suitable combination of the foregoing. More specific examples of the machine readable storage medium would include an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing.

Computer program code for carrying out methods of the present disclosure may be written in any combination of one or more programming languages. These computer program codes may be provided to a processor of a general purpose computer, special purpose computer, or other programmable data processing apparatus, such that the program codes, when executed by the processor of the computer or other programmable data processing apparatus, cause the functions or operations specified in the flowcharts and/or block diagrams to be implemented. The program code may execute entirely on a computer, partly on the computer, as a stand-alone software package, partly on the computer and partly on a remote computer or entirely on the remote computer or server.

Further, while operations are depicted in a particular order, this should not be understood as requiring that such operations be performed in the particular order shown or in sequential order, or that all illustrated operations be performed, to achieve desirable results. In certain circumstances, multitasking and parallel processing may be advantageous. Likewise, while several specific implementation details are contained in the above discussions, these should not be construed as limitations on the scope of any disclosure or of what may be claimed, but rather as descriptions of features that may be specific to particular embodiments of particular disclosures. Certain features that are described in this specification in the context of separate embodiments can also be implemented in combination in a single embodiment. Conversely, various features that are described in the context of a single embodiment can also be implemented in multiple embodiments separately or in any suitable sub-combination.

Various modifications, adaptations to the foregoing example embodiments of this disclosure may become apparent to those skilled in the relevant arts in view of the foregoing description, when read in conjunction with the accompanying drawings. Any and all modifications will still fall within the scope of the non-limiting and example embodiments of this disclosure. Furthermore, other embodiments of the disclosures set forth herein will come to mind to one skilled in the art to which these embodiments of the disclosure pertain having the benefit of the teachings presented in the foregoing descriptions and the drawings.

Therefore, it will be appreciated that the embodiments of the disclosure are not to be limited to the specific embodiments disclosed and that modifications and other embodiments are intended to be included within the scope of the appended claims. Although specific terms are used herein, they are used in a generic and descriptive sense only and not for purposes of limitation.

1. A control system for controlling a sleep monitoring device, comprising:
an input interface configured to receive a Ballistocardiography (BCG) signal;
an output interface for communicating with the sleep monitoring device; and
a processor coupled to the input interface and configured to:
determine, from a plurality of predefined sleep positions, a sleep position of a user of the sleep monitoring device based on the BCG signal;
generate a sequence of physiological parameters from the BCG signal;
adjust configuration for processing the sequence of physiological parameters based on the sleep position; and
control, via the output interface, the sleep monitoring device to monitor a sleep event of the user based on the sequence of physiological parameters and the adjusted configuration.

2. The control system of claim 1, wherein the processor is further configured to determine the sleep position of the user of the sleep monitoring device by:
detecting a peak in the BCG signal;
extracting at least one signal pattern from the BCG signal by capturing a portion of the BCG signal with at least one sample window, wherein a center of the at least one sample window is aligned with a time of the peak;
determining a set of correlations between the at least one signal pattern and a plurality of patterns for the plurality of predefined sleep positions; and
determining the sleep position corresponding to a maximum in the set of correlations.

3. The control system of claim 2, wherein each of the at least one sample window has a predefined size corresponding to a range of a physiological parameter.

4. The control system of claim 2, wherein determining, from the plurality of predefined sleep positions, the sleep position from the plurality of predefined sleep positions comprises:
determining a sample window of the at least one sample window corresponding to the maximum in the set of correlations;
detecting a further peak in the BCG signal;
extracting a signal pattern from the BCG signal by capturing a portion of the BCG signal with the sample window, wherein a center of the sample window is aligned with a time of the further peak;
determining a further set of correlations between the signal pattern and the plurality of patterns; and
determining the sleep position corresponding to a maximum in the further set of correlations.

5. The control system of claim 1, wherein the processor is further configured to:
filter the BCG signal with a predefined filtering approach to enhance features in the BCG signal; and
generate the sequence of physiological parameters based on the features in the filtered BCG signal.

6. The control system of claim 5, wherein the processor is further configured to:
select the predefined filtering approach based on the sleep position;
filter the BCG signal with the selected predefined filtering approach to enhance features in the BCG signal; and
generate the sequence of physiological parameters from the features in the filtered BCG signal.

7. The control system of 6, wherein the processor is further configured to generate the sequence of physiological parameters from the features in the filtered BCG signal by:
for each of a first set of sliding windows:
detecting features of the filtered BCG signal in the sliding window based on a feature detection manner; and
generating one or more values of the sequence of physiological parameters based on the detected features;
wherein the feature detection manner and a first stride between starting times of two adjacent sliding windows of the first set of sliding windows are adjustable based on the sleep position.

8. The control system of claim 1, wherein the processor is further configured to control the sleep monitoring device to monitor the sleep event of the user by:
determining a variability of the sequence of physiological parameters; and
responsive to the variability being lower than a predetermined threshold, determining an occurrence of the sleep event.

9. The control system of claim 1, wherein the processor is further configured to adjust the configuration for processing the sequence of physiological parameters by:
adjusting a second stride of a second set of sliding windows based on the sleep position, wherein the second stride indicates a difference between starting times of two adjacent sliding windows of the second set of sliding windows,
wherein the processor is further configured to control the sleep monitoring device to monitor the sleep event based on at least a portion of the sequence of physiological parameters within at least a part of the second set of sliding windows.

10. The control system of claim 9, wherein the processor is further configured to:
responsive to a detection of the sleep event by the sleep monitoring device, decrease the second stride between two adjacent sliding windows; and/or
responsive to no detection of the sleep event by the sleep monitoring device, increase the second stride between two adjacent sliding windows.

11. The control system of claim 9, wherein the processor is further configured to:
adjust a size of each of the second set of sliding windows based on the sequence of physiological parameters of the user.

12. The control system of claim 9, wherein the processor is further configured to:
determine occurrence number of the sleep event for one or more of the detected sleep positions during a period of time; and
adjust the second stride of the second set of sliding windows based on the occurrence number.

13. The control system of claim 1, wherein the processor is further configured to:
adjust a sampling frequency of a BCG sensor for sensing the BCG signal and/or a data storage manner of the BCG signal based on the sleep position.

14. A sleep monitoring system, comprising:
a Ballistocardiography (BCG) sensor configured to generate a BCG signal;
a sleep monitoring device configured to monitor a sleep event of a user; and
the control system of any of claim 1,
wherein the BCG sensor is communicatively coupled to an input interface of the control system, and the sleep monitoring device is communicatively coupled to an output interface of the control system.

15. A sleep monitoring device, comprising:
a processing unit; and
a memory having a plurality of instructions stored thereon, the plurality of instructions, when executed by the processing unit, cause the processing unit to:
determine, from a plurality of predefined sleep positions, a sleep position of a user of the sleep monitoring device based on a Ballistocardiography (BCG) signal;
generate a sequence of physiological parameters from the BCG signal;
adjust configuration for processing the sequence of the physiological parameters based on the sleep position; and
monitor a sleep event of the user based on the sequence of physiological parameters and the adjusted configuration.
